(19) United States
(12) Patent Application Publication
(10) Pub. No.: US 2009/0209492 A1
(43) Pub. Date: Aug. 20, 2009

(54) Lipase Inhibitors

(75) Inventors: William W. Bachovchin, Cambridge, MA (US); Hung-Sen Lai, Andover, MA (US); Daniel Philip O'Connell, Boston, MA (US)

Correspondence Address: FOLEY HOAG, LLP;PATENT GROUP, WORLD TRADE CENTER WEST, 155 SEAPORT BLVD, BOSTON, MA, 02110, US

(73) Assignee: TRUSTEES OF TUFTS COLLEGE, BOSTON, MA (US)

(21) Appl. No.: 11/719,159

(22) PCT Filed: Nov. 14, 2005
(86) PCT No.: PCT/US05/41022

§ 371 Date: Mar. 13, 2009

Related U.S. Application Data
(60) Provisional application No. 60/627,476, filed on Nov. 12, 2004.

(51) Int. Cl. A61K 31/69; C07F 5/02; A61P 3/00

(52) U.S. Cl. 514/64; 568/6

(57) ABSTRACT

The present invention relates to inhibitors of lipases, such as inhibitors of endothelial lipase, as well as pharmaceutical compositions thereof, and methods for using such inhibitors. The prototype of these inhibitors has lipophilic portion and an electrophilic site.

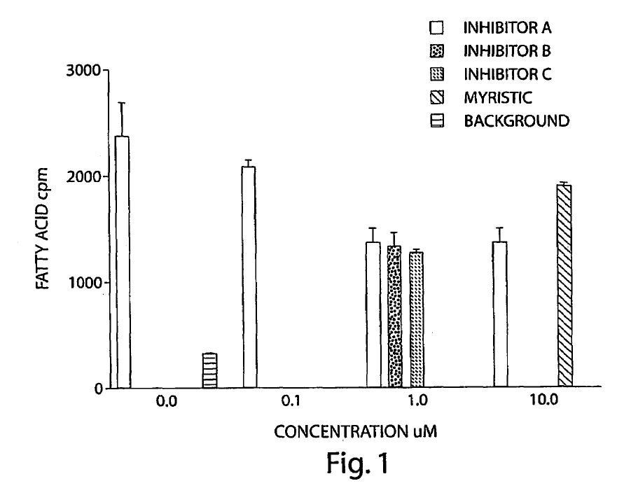

### BACKGROUND OF THE INVENTION

Cardiovascular disease is a major health risk throughout the industrialized world. Atherosclerosis, the most prevalent of cardiovascular diseases, is the principal cause of heart attack, and stroke, and thereby the principal cause of death in the United States. Atherosclerosis is a complex disease involving many cell types and molecular factors (for a detailed review, see Ross, 1993, Nature 362: 801-809). Results from epidemiologic studies have clearly established an inverse relationship between levels of high density lipoprotein (HDL), which transports endogenous cholesterol from tissues to the liver as well as mediating selective cholesteryl ester delivery to steroidogenic tissues, and the risk for atherosclerosis (Gordon and Rifkind, N. Engl. J. Med. 1989, 321, 1311-1316).

The metabolism of HDL is influenced by several members of the triacylglycerol (TG) lipase family of proteins, which hydrolyze triglycerides, phospholipids, and cholesteryl esters, generating fatty acids to facilitate intestinal absorption, energy production, or storage. Of the TG lipases, lipoprotein lipase (LPL) influences the metabolism of HDL cholesterol by hydrolyzing triglycerides in triglyceride-rich lipoproteins, resulting in the transfer of lipids and apolipoproteins to HDL and is responsible for hydrolyzing chylomicron and very low density lipoprotein (VLDL) in muscle and adipose tissues. Hepatic lipase (HL) hydrolyzes HDL triglyceride and phospholipids, generating smaller, lipid-depleted HDL particles, and plays a role in the uptake of HDL cholesterol (Jin et al., Trends Endocrinol. Metab., 2002, 13, 174-178; Wong and Schotz, J. Lipid Res., 2002, 43, 993-999). Endothelial lipase (also known as EDL, EL, LIPG, endothelial-derived lipase, and endothelial cell-derived lipase) is synthesized in endothelial cells, a characteristic that distinguishes it from the other members of the family.

At least 50% of the variation in HDL cholesterol levels is genetically determined. The phenotype of elevated HDL cholesterol is often dominantly inherited, but homozygous deficiency of HL or of the cholesteryl ester transfer protein (CETP), which result in elevated HDL cholesterol, are recessive conditions. Recently, several genetic variations in the human endothelial lipase gene have been identified, six of which potentially produce functional variants of the protein, and the frequencies of these variants were found to be associated with elevated levels of HDL cholesterol in human subjects (deLemos et al., Circulation, 2002, 106, 1321-1326). Notably, the endothelial lipase-mediated binding and uptake of HDL particles and the selective uptake of HDL-derived cholesterol esters have been reported to be independent of its enzymatic lipolytic activity (Strauss et al., Biochem. J., 2002).

Recombinant endothelial lipase protein has substantial phospholipase activity but has been reported to have less hydrolytic activity toward triglyceride lipids (Hirata et al., J. Biol. Chem., 1999, 274, 14170-14175; Jaye et al., Nat. Genet., 1999, 21, 424-428). However, endothelial lipase does exhibit triglyceride lipase activity ex vivo in addition to its HDL phospholipase activity, and endothelial lipase was found to hydrolyze HDL more efficiently than other lipoproteins (McCoy et al., J. Lipid Res., 2002, 43, 921-929). Overexpression of the human endothelial lipase gene in the livers of mice markedly reduces plasma concentrations of HDL cholesterol and its major protein apolipoprotein A-I (apoA-1) (Jaye et al., Nat. Genet., 1999, 21, 424-428).

Thus, there is a need for compounds that can inhibit lipase, particularly endothelial lipase, and are suitable for pharmaceutical use.

### SUMMARY OF THE INVENTION

One aspect of the invention relates to inhibitors of lipases, particularly triglyceride lipases including lipoprotein lipase, hepatic lipase, pancreatic lipase, and endothelial lipase having a structure of Formula I:

(R¹-L-R²)ₙ(CH₂)ₘ—X (I)

or a pharmaceutically acceptable salt thereof, where:

R¹ and R² are independently selected from C₁₋₆alkyl, C₁₋₆alkenyl, and C₁₋₆alkynyl;

R is selected from H, C₁₋₆alkyl, and C₁₋₆aralkyl;

L is absent or is selected from O, NR, and S;

X is a functional group that reacts with an active site residue of a targeted lipase to form a covalent adduct;

m is 0 or 1; and

n is an integer from 1-3.

A second aspect of the invention relates to inhibitors of lipases, particularly triglyceride lipases including lipoprotein lipase, hepatic lipase, pancreatic lipase, and endothelial lipase having a structure of Formula II:

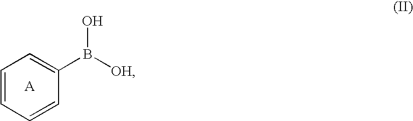

or a pharmaceutically acceptable salt thereof, where Ring A is optionally substituted by one or more functional groups.

Another aspect of the invention provides a pharmaceutical composition comprising a pharmaceutically acceptable carrier and one or more of the subject lipase inhibitors, or a pharmaceutically acceptable salt or prodrug thereof.

Another aspect of the invention provides for use of one or more of the subject inhibitors in the manufacture of a medicament for inhibiting a lipase in vivo. In certain embodiments, the subject inhibitors may be used in the manufacture of a medicament for increasing plasma concentrations of HDL. In certain embodiments, the subject inhibitors may be used in the manufacture of a medicament for the treatment of a disease or condition such a vascular disease or condition. In certain embodiments the vascular disease or condition is a cardiovascular disease or condition selected from angina, atherosclerosis, coronary artery disease, congestive heart failure, hypertension, myocardial infarction, and stroke.

Another aspect of the invention relates to a method for increasing plasma concentrations of HDL. In certain embodiments, the invention relates to a method for the treatment of a vascular disease or condition, comprising administering an inhibitor of the invention to a subject. In certain embodiments the vascular disease or condition is a cardiovascular disease or condition or condition selected from angina, atherosclerosis, coronary artery disease, congestive heart failure, hypertension, myocardial infarction, and stroke.

Yet another aspect of the invention provides a packaged pharmaceutical comprising: a preparation of one or more of the subject lipase inhibitors; a pharmaceutically acceptable carrier; and instructions, written and/or pictorial, describing the use of the preparation for inhibiting a lipase in vivo, such as for regulating HDL metabolism.

### BRIEF DESCRIPTION OF THE FIGURES

The FIGURE shows the effect of inhibitor compounds A-C compared to the effect of myristic acid on endothelial lipase activity.

### DETAILED DESCRIPTION OF THE INVENTION

The present invention relates to inhibitors of lipases, such as inhibitors of endothelial lipase, as well as pharmaceutical compositions thereof, and methods for using such inhibitors. The prototype of these molecules has a lipophilic portion and an electrophilic site.

The compounds of the present invention can be used as part of treatments for a variety of diseases or conditions, such as those that are mediated by endothelial lipase. For instance, the subject inhibitors can be used to regulate HDL metabolism, and more generally, the subject inhibitors may be used for the treatment of a vascular disease or condition.

One aspect of the invention relates to lipase inhibitors having a structure of Formula I

(R¹-L-R²)ₙ(CH₂)ₘ—X (I)

where:

R¹ and R² are independently selected from C₁₋₆alkyl, C₁₋₄alkenyl, and C₁₋₄alkynyl;

R is selected from H, C₁₋₆alkyl, and C₁₋₆aralkyl

L is absent or is selected from O, NR, and S;

X is a functional group that reacts with an active site residue of the targeted lipase to form a covalent adduct;

m is 0 or 1; and

n is an integer from 1-3.

In certain embodiments, R¹ and R² are independently C₁₋₄ alkyl. In certain such embodiments, L is absent. In preferred such embodiments, L is absent, n is 1 or 2, and R¹ and R² are independently C₁₋₆alkyl.

Preferably, R¹, R², m and n are selected such that there are from 7 to 16, 7 to 15 or 7 to 14 carbon atoms. The resulting combination of alkyl and alkenyl groups is typically either saturated (i.e., all alkyl) or has a single double bond. When a double bond is present, it is typically located at the distal end of the chain (i.e., away from X) or directly adjacent to X. Such groups are typically unsubstituted.

In certain embodiments, L is absent, m is 0, n is 2, R¹ is 3 or 4, and R² is 4. In certain embodiments, L is absent, m is 1, n is 2, R¹ is 3 or 4, and R² is 4.

In certain embodiments, X is selected from boronic acid or a group that is hydrolysable to boronic acid, —CN, —SO₂Z¹, —P(═O)Z¹, —P(═R³)R⁴R⁵, —C(═NH)NH₂, —CH═NR⁶, and —C(═O)—R⁶ wherein:

R³ is O or S;

R⁴ is selected from N₃, SH₂, NH₂, NO₂, and OYR⁷, and

R⁵ is selected from lower alkyl, amino, OYR⁷, and a pharmaceutically acceptable salt thereof, or

R⁴ and R⁵, together with the phosphorus to which they are attached, form a 5- to 8-membered heterocyclic ring;

R⁶ is selected from H, alkyl, alkenyl, allynyl, —NH₂, —(CH₂)ₚ—R⁷, —(CH₂)_{q}—OH, ——(CH₂)_{q}—O-alkyl, —(CH₂)_{q}—O-alkenyl, —(CH₂)_{q}—O-alkynyl, —(CH₂)_{q}—O—(CH₂)ₚ—R⁷, —(CH₂)_{q}—SH, —(CH₂)_{q}—S-alkyl, —(CH₂)_{q}—S-alkenyl, —(CH₂)_{q}—S-alkynyl, —(CH₂)_{q}—S—(CH₂)ₚ—R⁷, —C(O)NH₂, —C(O)OR⁸, and C(Z¹)(Z²)(Z³);

R⁷ is selected from H, alkyl, alkenyl, aryl, heteroaryl, cycloalkyl, cycloalkenyl, and heterocyclyl;

R⁸ is selected from H, alkyl and alkenyl;

Y is absent or is selected from alkyl, alkenyl, alkynyl, —(CH₂)ᵣ(OCH₂)ᵣ, —(CH₂)ᵣNR₂(CH₂)ᵣ—, and —(CH₂)ₙS(CH₂)ᵣ—;

Z¹ is a halogen;

Z² and Z³ are independently selected from H and halogen;

p is, independently for each occurrence, an integer from 0 to 8;

q is, independently for each occurrence, an integer from 1 to 8; and

r is, independently for each occurrence, an integer from 0 to 10.

In certain embodiments, X is selected from CN, CHO, and C(═O)C(Z¹)(Z²)(Z³), wherein Z¹ is a halogen, and Z² and Z³ are independently selected from H or halogen. In another embodiment, X is C(═O)C(Z¹)(Z²)(Z³), wherein Z¹ is fluorine, and Z² and Z³ represent H or fluorine.

In certain preferred embodiments, X is a group of formula —B(Y¹)(Y²), wherein Y¹ and Y² are independently —OH or —B(Y¹)(Y²) is hydrolysable to a boronic acid, such as a 5- to 8-membered ring that is hydrolysable to a boronic acid.

Another aspect of the invention relates to lipase inhibitors having the structure of Formula II:

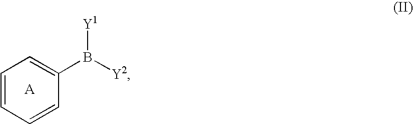

or a pharmaceutically acceptable salt thereof, where:

Ring A is optionally substituted by one or more functional groups; and

—B(Y¹)(Y²) is B(OH)₂ or a group that is hydrolysable to B(OH)₂, such as a 5- to 8-membered ring that is hydrolysable to a boronic acid.

Preferably, Ring A is substituted by at least one alkyl group. Typically, one alkyl group is unsubstituted or substituted by an oxo group (e.g., an acetyl group). When more than one alkyl group substituent is present, second and further alkyl groups are advantageously substituted with groups that interact with lipase (e.g., endothelial lipase), such as at or near the active site. Suitable substituents on alkyl groups include carboxylate, ester, amide, amino, hydroxyl and thiol groups. These substituents, along with halogens can also be directly substituted on Ring A.

In certain embodiments, compounds of Formula II are represented by Formula III:

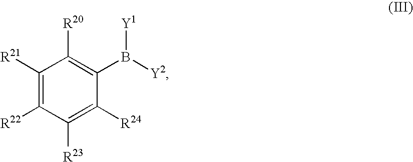

or a pharmaceutically acceptable salt thereof, where:

R²⁰, R²¹, R²³ and R²⁴ are each independently —H, —COOR′, —CONR′R″, —C(O)R′, —NR′R″, —OH, —SH or a alkyl, alkenyl or alkynyl group optionally substituted by one or more of —COOR′, —CONR′R″, —C(O)R′, —NR′R″, —OH and —SH;

R²² is an unsubstituted C₁₋₁₂ alkyl group or an oxo-substituted C₁₋₁₂ alkyl group;

R′ and R″ are each independently —H or an alkyl, alkenyl, alkynyl, aryl or heteroaryl group; and

BY¹Y² is B(OH)₂ or a group that is hydrolysable to B(OH)₂, such as a 5- to 8-membered ring that is hydrolysable to a boronic acid.

In certain embodiments, three of R²⁰, R²¹, R²³ and R²⁴ are —H. In certain such embodiments, the remaining one of R²⁰, R²¹, R²³ and R²⁴ is an alkyl, alkenyl or alkynyl group optionally substituted by one or more of —COOR′, —CONR′R″, —C(O)R′, —NR′R″, —OH and —SH, particularly —COOH.

In certain embodiments, such as when R²⁰, R²¹, R²³ and R²⁴ have the values described above, R is an unsubstituted C₁₋₈ alkyl group.

In certain embodiments, the lipase inhibitor inhibits endothelial lipase with a Kᵢ of 50 nm or less.

In certain embodiments, the inhibitor is orally active.

In certain embodiments, the inhibitor has a therapeutic index in humans of at least 2, and even more preferably 5, 10 or even 100, e.g., such as a therapeutic index for regulating HDL metabolism.

Another aspect of the invention provides a pharmaceutical composition comprising a pharmaceutically acceptable carrier and one or more of the subject lipase inhibitors, or a pharmaceutically acceptable salt or prodrug thereof.

Another aspect of the invention provides for use of one or more of the subject inhibitors in the manufacture of a medicament for inhibiting a lipase in vivo. In certain embodiments, the subject inhibitors may be used in the manufacture of a medicament for increasing plasma concentrations of HDL (e.g., as part of treatment for metabolic syndrome or Syndrome X). In certain embodiments, the subject inhibitors may be used in the manufacture of a medicament for the treatment of a disease or condition such a vascular disease or condition. In certain embodiments the vascular disease or condition is a cardiovascular disease or condition selected from angina, atherosclerosis, coronary artery disease, congestive heart failure, hypertension, myocardial infarction, and stroke.

Another aspect of the invention relates to a method for increasing plasma concentrations of HDL. In certain embodiments, the invention relates to a method for the treatment of a vascular disease or condition, comprising administering an inhibitor of the invention. In certain embodiments, the vascular disease or condition is a cardiovascular disease or condition or condition selected from angina, atherosclerosis, coronary artery disease, congestive heart failure, hypertension, myocardial infarction, and stroke.

Another aspect of the invention provides a conjoint therapy wherein one or more other therapeutic agents are administered with the lipase inhibitor. Such conjoint treatment may be achieved by way of the simultaneous, sequential, or separate dosing of the individual components of the treatment.

In one embodiment, an inhibitor(s) is conjointly administered with an anti-dyslipidemic agent. Anti-dyslipidemic agent useful in the compositions of the present invention include (1) bile acid sequestrants, (2) HMG-CoA reductase inhibitors, (3) HMG-CoA synthase inhibitors, (4) cholesterol absorption inhibitors, (5) acyl coenzyme A-cholesterol acyl transferase (ACAT) inhibitors, (6) cholesteryl ester transfer protein (CETP) inhibitors, (7) squalene synthetase inhibitors, (8) anti-oxidants, (9) PPAR alpha agonists, (10) FXR receptor antagonists, (I 1) LXR receptor agonists, (12) lipoprotein synthesis inhibitors, (13) renin angiotensin system inhibitors, (14) microsomal triglyceride transport inhibitors, (15) bile acid reabsorption inhibitors, (16) PPAR gamma agonists, (17) triglyceride synthesis inhibitors, (18) transcription modulators, (19) squalene epoxidase inhibitors, (20) low density lipoprotein (LDL) receptor inducers, (21) platelet aggregation inhibitor, (22) 5-LO or FLAP inhibitors, (23) PPAR partial agonists, and (24) niacin or a niacin receptor agonists, and pharmaceutically acceptable salts and esters thereof.

Bile acid sequestrants include cholestyramine, colestipol, dialkylaminoalkyl derivatives of a cross-linked dextran, colesevelam, sevelamer and pharmaceutically acceptable salts and esters thereof.

HMG-CoA reductase inhibitors include atorvastatin, cerivastatin, itavastatin, fluvastatin, lovastatin, pravastatin, rivastatin, simvastatin, rosuvastatin, and ZD-4522 and pharmaceutically acceptable salts and esters thereof.

Cholesterol absorption inhibitors include beta-sitosterol, ezetimibe, and tiqueside and pharmaceutically acceptable salts and esters thereof.

Acyl coenzyme A-cholesterol acyl transferase (ACAT) inhibitors include avasimibe, eflucimibe, KY505 and SMP 797 and pharmaceutically acceptable salts and esters thereof.

Anti-oxidants include probucol and pharmaceutically acceptable salts and esters thereof.

PPAR alpha agonists include fibrates such as beclofibrate, benzafibrate, ciprofibrate, clofibrate, etofibrate, fenofibrate, clinofibrate and gemfibrozil and pharmaceutically acceptable salts and esters thereof.

Lipoprotein synthesis inhibitors include niacin or nicotinic acid and nicotinamide and pharmaceutically acceptable salts and esters thereof.

In another embodiment, an inhibitor(s) is conjointly administered with another drug(s) commonly used to treat lipid disorders. Such drugs include, but are not limited to, thiazolidinediones (e.g., glitazones), cholesterol ester transfer inhibitors, apoA1 mimetics (e.g., L-4F), apoB-secretion inhibitors, and MTP inhibitors. Examples of glitazones include triglitazone, pioglitazone and rosiglitazone. Examples of MTP inhibitors include BMS-201038 (9-[4-[4-[2-(4-trifluoromethylphenyl)benzoylamino]piperidin-1-yl]butyl]-N-(2,2,2-trifluoro-ethyl)-9H-fluorene-9-carboxamide) and CP-346086 (4′-trifluoromethyl-biphenyl-2-carboxylic acid [2-(2H-[1,2,4]triazol-3-ylmethyl)-1,2,3,4-tetrahydro-isoquinolin-6-yl]amide).

In a further embodiment of the invention, an inhibitor(s) is conjointly administered with another drug(s) commonly used to treat diabetes. Examples of such drugs include insulin, DPIV inhibitors (e.g., boronic acids, agents disclosed in U.S. Pat. Nos. 5,462,928, 6,803,357, 6,825,169, 6,890,898, U.S. Publication Nos. 2003/0153509, 2004/0176307, 2004/0229820 and 2005/0203027, and International Publication Nos. WO 2005/082849 and WO 2005/082348, the contents of which are incorporated herein by reference), GLP-1 and analogs thereof (e.g., exendins such as exendin-4), peptide hormones (e.g., GLP-2, GIP, or NPY), gene therapy vectors which cause the ectopic expression of said agents and peptide hormones, variants of a naturally occurring or synthetic peptide hormone where one or more amino acids have been added, deleted, or substituted, Ml receptor antagonists, and cholinergic agents (e.g., substances that directly or indirectly block activation of muscarinic cholinergic receptors such as quaternary amines (methantheline, ipratropium, and propantheline), tertiary amines (dicyclomine and scopolamine), and tricyclic amines (telenzepine), particularly pirenzepine and methyl scopolamine). Other suitable muscarinic receptor antagonists include benztropine (commercially available as COGENTIN from Merck), hexahydro-sila-difenidol hydrochloride; (+/−)-3-quinuclidinyl xanthene-9-carboxylate hemioxalate (QNX-hemioxalate), telenzepine dihydrochloride, and atropine. Additional examples of drugs for the treatment of diabetes include prolactin inhibitors such as d2 dopamine agonists (e.g. bromocriptine), prolactin-inhibiting ergo alkaloids and prolactin-inhibiting dopamine agonists (e.g., 2-bromo-alpha-ergocriptine, 6-methyl-8 beta-carbobenzyloxyaminoethyl-10-alpha-ergoline, 8-acylaminoergolines, 6-methyl-8-alpha-(N-acyl)amino-9-ergoline, 6-methyl-8-alpha-(N-phenylacetyl)amino-9-ergoline, ergocornine, 9,10-dihydroergocornine, D-2-halo-6-alkyl-8-substituted ergolines, D-2-bromo-6-methyl-8-cyanomethylergoline, carbidopa, benserazide, and other dopadecarboxylase inhibitors, L-dopa, dopamine, and non toxic salts thereof). Further examples of drug for the treatment of diabetes are those which act on the ATP-dependent potassium channel of the β-cells (e.g., glibenclamide, glipizide, gliclazide, AG-EE 623 ZW), metformin and related compounds, and glucosidase inhibitors (e.g., acarbose).

Inhibitors of the invention are also useful as part as hormone replacement therapy (e.g., with estrogen, progestin, combinations thereof, etc.) in women, in order to enhance the cardioprotective effects of such therapies.

Yet another aspect of the invention provides a packaged pharmaceutical comprising: a preparation of one or more of the subject lipase inhibitors; a pharmaceutically acceptable carrier; and instructions, written and/or pictorial, describing the use of the preparation for inhibiting a lipase in vivo, such as for regulating HDL metabolism.

The packaged pharmaceutical can also include, e.g., as co-formulation the lipase inhibitor or simply co-packaged with one or more agents listed above, such as an HMG-CoA reductase inhibitor (such as lovastatin, simvastatin, pravastatin, fluvastatin, or atorvastatin), a bile acid sequestrant (such as cholestyramine, colestipol, or colesevelam), nicotinic acid, a fibrate (such as gemfibrozil), ezetimide, bile acid sequestrants, glitazones, MTP inhibitors, ACAT inhibitors, CETP inhibitors, agents used in hormone replacement therapy and/or agents that cause unwanted changes in HDL levels as a side effect.

In certain preferred embodiments, the method involves administration of a lipase inhibitor, preferably at a predetermined time(s) during a 24-hour period, in an amount effective to improve one or more aberrant indices associated with atherosclerosis.

### DEFINITIONS

The term “co-formulation” or “co-formulate” as used herein refers to two or more therapeutic agents that are components of a single composition such as a solution, tablet, pill, etc.

The term “co-package” as used herein refers to two or more therapeutic agents that are separately formulated (not co-mingled) contained in the same end-user packaging. A co-package may include, for example, a solution of therapeutic agent A in bottle A and a solution of therapeutic agent B in bottle B packaged together in a single kit.

The term “C_{x-y} alkyl” refers to optionally substituted saturated hydrocarbon groups, including straight-chain alkyl and branched-chain alkyl groups that contain from x to y carbons in the chain. “Lower alkyl” groups have from 1 to 4 carbon atoms. The terms “C_{2-y}alkenyl” and “C_{2-y}alkynyl” refer to substituted or unsubstituted unsaturated aliphatic groups analogous in length and possible substitution to the alkyls described above, but that contain at least one double or triple bond respectively.

The term “aryl” refers to an aromatic hydrocarbon ring system. Aromatic rings are monocyclic or fused bicyclic ring systems, such as phenyl, naphthyl, etc. Monocyclic aromatic rings contain from about 5 to about 10 carbon atoms, preferably from 5 to 7 carbon atoms, and most preferably from 5 to 6 carbon atoms in the ring. Bicyclic aromatic rings contain from 8 to 12 carbon atoms, preferably 9 or 10 carbon atoms in the ring. The term “aryl” also includes bicyclic ring systems wherein only one of the rings is aromatic, e.g., the other ring is cycloalkyl, cycloalkenyl, or heterocyclyl. Aromatic rings may be unsubstituted or substituted with from 1 to about 5 substituents on the ring.

The term “heteroaryl” refers to an aromatic ring system containing carbon and from 1 to about 4 heteroatoms in the ring. Heteroaromatic rings are monocyclic or fused bicyclic ring systems. Monocyclic heteroaromatic rings contain from about 5 to about 10 member atoms (carbon and heteroatoms), preferably from 5 to 7, and most preferably from 5 to 6 in the ring. Bicyclic heteroaromatic rings contain from 8 to 12 member atoms, preferably 9 or 10 member atoms in the ring. The term “heteroaryl” also includes bicyclic ring systems where only one of the rings is aromatic, e.g., the other ring is cycloalkyl, cycloalkenyl, or heterocyclyl. Heteroaromatic rings may be unsubstituted or substituted with from 1 to about 4 substituents on the ring. Exemplary heteroaromatic rings include thienyl, thiazolyl, oxazolyl, pyrrolyl, purinyl, pyrimidyl, pyridyl, and furanyl.

Suitable substituents for alkyl, alkenyl, alkynyl, aryl and heteroaryl groups include, for example, an alkyl, an alkenyl, an alkynyl, an aryl, a heteroaryl, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, a cyano, a nitro, a sulfhydryl, an alkylthio, a sulfate, a sulfonate, a sulfamoyl, a sulfonamido, a sulfonyl, a heterocyclyl, an aralkyl, or an aromatic or heteroaromatic moiety. Electrophilic groups such as aldehydes are typically not substituents in compounds of the invention.

As used herein, the term “inhibitor” is meant to describe a compound that blocks or reduces an activity of an enzyme. An inhibitor can act with competitive, uncompetitive, or noncompetitive inhibition. An inhibitor can bind reversibly or irreversibly, and therefore the term includes compounds that are suicide substrates of an enzyme. An inhibitor can modify one or more sites on or near the active site of the enzyme, or it can cause a conformational change elsewhere on the enzyme.

A “patient” or “subject” to be treated by the subject method can mean either a human or non-human subject. A patient r, subject in need of treatment for a disease or condition is a patient or subject who has a pathophysiological condition or a condition that can evolve into a pathophysiological condition that can be treated by administration of a therapeutically effective amount of a compound of the invention.

The term “IC₅₀” means the dose of a drug that inhibits a biological activity by 50%, e.g., the amount of inhibitor required to inhibit at least 50% of endothelial lipase (or another lipase) activity in vivo.

The term “prodrug” is intended to encompass compounds that, under physiological conditions, are converted into the therapeutically active agents of the present invention. A common method for making a prodrug is to include selected moieties that are hydrolyzed under physiological conditions to reveal the desired molecule. In other embodiments, the prodrug is converted by an enzymatic activity of the host animal.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “preventing” is art-recognized, and when used in relation to a condition, such as a local recurrence (e.g., pain), a disease such as a syndrome complex such as heart failure or any other medical condition, is well understood in the art, and includes administration of a composition which reduces the frequency of, or delays the onset of, symptoms of a medical condition in a subject relative to a subject which does not receive the composition. Prevention of a vascular disease or condition includes, for example, reducing the number of diagnoses of the vascular disease or condition in a treated population versus an untreated control population, and/or delaying the onset of symptoms of the vascular disease or condition in a treated population versus an untreated control population. Prevention of an infection includes, for example, reducing the number of diagnoses of the infection in a treated population versus an untreated control population, and/or delaying the onset of symptoms of the infection in a treated population versus an untreated control population. Prevention of pain includes, for example, reducing the magnitude of, or alternatively delaying, pain sensations experienced by subjects in a treated population versus an untreated control population.

A “therapeutically effective amount” of a compound, e.g., such as a lipase inhibitor of the present invention, with respect to the subject method of treatment, refers to an amount of the compound(s) in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

The term “vascular disease or condition” as used herein refers to any disease or condition effecting the vascular system, including the heart and blood vessels. A vascular disease or condition includes any disease or condition characterized by vascular dysfunction, including, for example, intravascular stenosis (narrowing) or occlusion (blockage), due to the development of atherosclerotic plaque and diseases and disorders resulting therefrom. Examples of vascular diseases and conditions include, without limitation, atherosclerosis, coronary artery disease (CAD), myocardial infarctions (MI), angina, ischemia, stroke, peripheral vascular diseases, venous thromboembolism, and pulmonary embolism.

### Pharmaceutical Compositions

Inhibitors prepared as described herein can be administered in various forms, depending on the disorder to be treated and the age, condition, and body weight of the patient, as is well known in the art. For example, where the compounds are to be administered orally, they may be formulated as tablets, capsules, granules, powders, or syrups; or for parenteral administration, they may be formulated as injections (intravenous, intramuscular, or subcutaneous), drop infusion preparations, or suppositories. For application by the ophthalmic mucous membrane route, they may be formulated as eye drops or eye ointments. These formulations can be prepared by conventional means, and, if desired, the active ingredient may be mixed with any conventional additive, such as an excipient, a binder, a disintegrating agent, a lubricant, a corrigent, a solubilizing agent, a suspension aid, an emulsifying agent, or a coating agent. Although the dosage will vary depending on the symptoms, age and body weight of the patient, the nature and severity of the disorder to be treated or prevented, the route of administration and the form of the drug, in general, a daily dosage of from 0.001 to 200 mg of the compound is recommended for an adult human patient, and this may be administered in a single dose or in divided doses.

The precise time of administration and/or amount of the inhibitor that will yield the most effective results in terms of efficacy of treatment in a given patient will depend upon the activity, pharmacokinetics, and bioavailability of a particular compound, physiological condition of the patient (including age, sex, disease type and stage, general physical condition, responsiveness to a given dosage, and type of medication), route of administration, etc. However, the above guidelines can be used as the basis for fine-tuning the treatment, e.g., determining the optimum time and/or amount of administration, which will require no more than routine experimentation consisting of monitoring the subject and adjusting the dosage and/or timing.

The phrase “pharmaceutically acceptable” is employed herein to refer to those ligands, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose, and sucrose; (2) starches, such as corn starch and potato starch; (3)-cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar, (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. In certain embodiments, pharmaceutical compositions of the present invention are non-pyrogenic, i.e., do not induce significant temperature elevations when administered to a patient.

The term “pharmaceutically acceptable salts” refers to the relatively non-toxic, inorganic and organic acid addition salts of the inhibitor(s). These salts can be prepared in situ during the final isolation and purification of the inhibitor(s), or by separately reacting a purified inhibitor(s) in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts, and the like. (See, for example, Berge et al. (1977) “Pharmaceutical Salts”, J. Pharm. Sci. 66:1-19)

In other cases, the inhibitors useful in the methods of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic inorganic and organic base addition salts of an inhibitor(s). These salts can likewise be prepared in situ during the final isolation and purification of the inhibitor(s), or by separately reacting the purified inhibitor(s) in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like (see, for example, Berge et al., supra).

Wetting agents, emulsifiers, and lubricants, such as sodium lauryl sulfate and magnesium stearate, as well as coloring agents, release agents, coating agents, sweetening, flavoring, and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite, and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations useful in the methods of the present invention include those suitable for oral, nasal, topical (including buccal and sublingual), rectal, vaginal, aerosol, and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated and the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an inhibitor(s) with the carrier and, optionally, one or more accessory ingredients. In general, the formulations are prepared by uniformly and intimately bringing into association a ligand with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouthwashes, and the like, each containing a predetermined amount of an inhibitor(s) as an active ingredient. A compound may also be administered as a bolus, electuary, or paste.

In solid dosage forms for oral administration (capsules, tablets, pills, dragees, powders, granules, and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets, and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols, and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered peptide or peptidomimetic moistened with an inert liquid diluent.

Tablets, and other solid dosage forms, such as dragees, capsules, pills, and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes, and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as, for example, water or other solvents, solubilizing agents, and emulsifiers such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols, and fatty acid esters of sorbitan, and mixtures thereof.

Besides inert diluents, the oral compositions can also include adjuvants such as wetting agents, emulsifying and suspending agents, sweetening, flavoring, coloring, perfuming, and preservative agents.

Suspensions, in addition to the active inhibitor(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more inhibitor(s) with one or more suitable nonirritating excipients or carriers comprising, for example, cocoa butter, polyethylene glycol, a suppository wax or a salicylate, which is solid at room temperature, but liquid at body temperature and, therefore, will melt in the rectum or vaginal cavity and release the active agent.

Formulations which are suitable for vaginal administration also include pessaries, tampons, creams, gels, pastes, foams, or spray formulations containing such carriers as are known in the art to be appropriate.

Dosage forms for the topical or transdermal administration of an inhibitor(s) include powders, sprays, ointments, pastes, creams, lotions, gels, solutions, patches, and inhalants. The active component may be mixed under sterile conditions with a pharmaceutically acceptable carrier, and with any preservatives, buffers, or propellants which may be required.

The ointments, pastes, creams, and gels may contain, in addition to inhibitor(s), excipients, such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to an inhibitor(s), excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane.

Herein, administration by inhalation may be oral and/or nasal. Examples of pharmaceutical devices for aerosol delivery include metered dose inhalers (MDIs), dry powder inhalers (DPIs), and air-jet nebulizers. Exemplary nucleic acid delivery systems by inhalation which can be readily adapted for delivery of the subject inhibitor(s) are described in, for example, U.S. Pat. Nos. 5,756,353 and 5,858,784 and PCT applications WO98/31346, WO98/10796, WO00/27359, WO01/54664, and WO02/060412. Other aerosol formulations that may be used for delivering the inhibitor(s) are described in U.S. Pat. Nos. 6,294,153, 6,344,194, and 6,071,497 and PCT applications WO02/066078, WO02/053190, WO01/60420, and WO00/66206.

In preferred embodiments, particularly where systemic dosing of the inhibitor(s) is desired, the aerosoled inhibitor(s) is formulated as microparticles. Microparticles having a diameter of between 0.5 and ten microns can penetrate the lungs, passing through most of the natural barriers. A diameter of less than ten microns is required to bypass the throat; a diameter of 0.5 microns or greater is required to avoid being exhaled.

Ordinarily, an aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars, or sugar alcohols. Aerosols generally are prepared from isotonic solutions.

Transdermal patches have the added advantage of providing controlled delivery of an inhibitor(s) to the body. Such dosage forms can be made by dissolving or dispersing the agent in the proper medium. Absorption enhancers can also be used to increase the flux of the inhibitor(s) across the skin. The rate of such flux can be controlled by either providing a rate controlling membrane or dispersing the peptidomimetic in a polymer matrix or gel.

Pharmaceutical compositions of this invention suitable for parenteral administration comprise one or more inhibitors(s) in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of inhibitor(s) in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the inhibitors(s) of the present invention are administered as pharmaceuticals to humans and animals, they can be given per se or as a pharmaceutical composition containing, for example, 0.1 to 99.5% (more preferably, 0.5 to 90%) of active ingredient in combination with a pharmaceutically acceptable carrier.

The preparations of agents may be given orally, parenterally, topically, or rectally. They are of course given by forms suitable for each administration route. For example, they are administered in tablets or capsule form, by injection, inhalation, eye lotion, ointment, suppository, infusion; topically by lotion or ointment; and rectally by suppositories. Oral administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection, and infusion.

The phrases “systemic administration,” “administered systemically,” “peripheral administration” and “administered peripherally” as used herein mean the administration of a ligand, drug, or other material other than directly into the central nervous system, such that it enters the patient's system and thus, is subject to metabolism and other like processes, for example, subcutaneous administration.

These inhibitors(s) may be administered to humans and other animals for therapy by any suitable route of administration, including orally, nasally, as by, for example, a spray, rectally, intravaginally, parenterally, intracisternally, and topically, as by powders, ointments or drops, including buccally and sublingually.

Regardless of the route of administration selected, the inhibitor(s), which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

### Exemplification

The invention now being generally described, it will be more readily understood by reference to the following examples which are included merely for purposes of illustration of certain aspects and embodiments of the present invention, and are not intended to limit the invention.

### Tetradecylboronic Acid

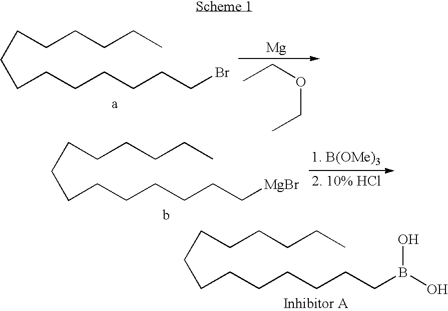

Magnesium turnings (2.84 g) were added to a dry 3-necked round bottom flask equipped with a reflux condenser that had been flushed with argon. Dry ether (5.0 mL) and one crystal of iodine were then added. 1-Bromotetradecane b (10 mL, 10.16 g, 36.6 mmol) were dissolved in ether (12 mL) and added over one hour to the magnesium. After complete addition of the bromide, the solution was allowed to stir for 2 hours without external heating.

A dry round bottom flask cooled to −78° C. equipped with a pressure equalizing addition funnel was flushed with argon followed by the addition of dry ether (90 mL) and trimethylborate (4.2 mL, 37 mmol). Compound b was then added dropwise over one hour. The solution was stirred for an additional hour before removing the cold bath and allowing the reaction to warm to room temperature. 10% HCl (50 mL) was then added dropwise to the reaction flask at room temperature. After 15 minutes of additional stirring, the biphasic solution was extracted into ether. The ethereal solutions were dried over MgSO₄ and the ether removed under reduced pressure. The resulting white solid was purified as follows by adding water (90° C.) to dissolve the products, the solution was then cooled to 4° C. to allow the boronic acid to precipitate as a white solid. This was filtered, the solid collected, and then washed with hexanes (60° C.). The flask was placed in the freezer for 1 hour. The precipitate was filtered, collected, and dried under vacuum to provide Inhibitor A.

### II. Hexadecylboronic Acid

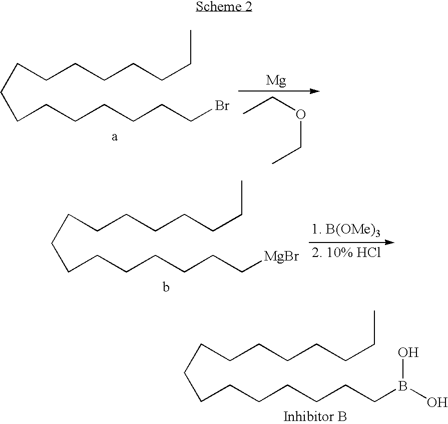

Magnesium turnings (2.55 g) were added to a dry 3-necked round bottom flask equipped with a reflux condenser that had been flushed with argon. Dry ether (4.5 mL) was added followed by one crystal of iodine. 1-Bromohexadecane (10 mL, 10.0 g, 32.75 mmol) was dissolved into 11 mL ether and over one hour to the magnesium under ether. After complete addition of the bromide, the solution was allowed to stir for 2 hours without external heating.

A dry 250 mL round bottom flask cooled to −78° C. equipped with a pressure equalizing addition funnel was flushed with argon and dry ether (80 mL) was added to the flask followed by addition of trimethylborate (3.75 mL, 33 mmol). Compound b was then added dropwise over one hour. The solution was stirred for an additional hour before removing the cold bath and allowing the reaction to warm to room temperature. 10% HCl (50 mL) was then added dropwise to the reaction flask at room temperature. After 15 minutes of additional stirring, the biphasic solution was extracted into ether. The ethereal solutions were dried over MgSO₄ and the ether removed under reduced pressure. The resulting white solid was purified as follows by adding water (90° C.) to dissolve the products, the solution was then cooled to 4° C. to allow the boronic acid to precipitate as a white solid. This was filtered, the solid collected, and then washed with hexanes (60° C.). The flask was placed in the freezer for 1 hour. The precipitate was filtered, collected, and dried under vacuum to provide Inhibitor B.

### III. Pentadecylboronic Acid

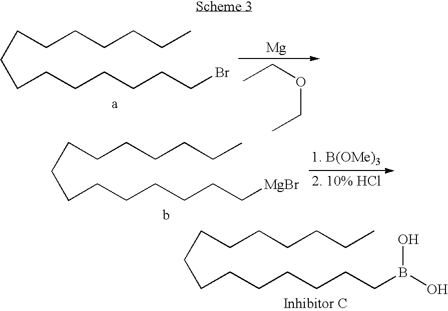

To a dry 3-necked round bottom flask equipped with a reflux condenser flushed with argon was added magnesium turnings (1.35 g) followed by addition of 2.3 mL of dry ether and one crystal of iodine. 1-Bromopentadecane a (5 mL, 5.0 g, 17.3 mmol) was dissolved into 6 mL ether and slowly added over one hour to the magnesium under ether. After complete addition of the bromide, the solution was allowed to stir for 2 hours without external heating.

A dry 250 mL round bottom flask cooled to −78° C. equipped with a 250 mL pressure equalizing addition funnel was flushed with argon followed by addition of 40 mL dry ether and 2.0 mL (17.5 mmol) trimethylborate. Compound b was then added dropwise over one hour. The solution was stirred for an additional hour before removing the cold bath and allowing the reaction to warm to room temperature. 10% HCl (50 mL) was then added dropwise to the reaction flask at room temperature. After 15 minutes of additional stirring, the biphasic solution was extracted into ether. The ethereal solutions were dried over MgSO₄ and the ether removed under reduced pressure. The resulting white solid was purified as follows by adding water (90° C.) to dissolve the products, the solution was then cooled to 4° C. to allow the boronic acid to precipitate as a white solid. This was filtered, the solid collected, and then washed with hexanes 60° C.). The flask was placed in the freezer for 1 hour. The precipitate was filtered, collected, and dried under vacuum to provide Inhibitor C.

### IV. Lipase Inhibiting Activity of Aliphatic Boronic Acids

The endothelial lipase inhibiting activity of various aliphatic boronic acids was tested. The results of the assay, which used a 50 micromolar solution of each compound, are shown below. The IC₅₀ of certain compounds for endothelial lipase was also obtained. A comparison of the endothelial lipase activity of n-C₁₄H₂₉B(OH)₂, n-C₁₅H₃₁B(OH)₂, n-C₁₆H₃₃B(OH)₂ and myristic acid is shown in the FIGURE.

| Compound | % Lipase Inhibition | IC₅₀ (micromolar) |
|---|---|---|
| 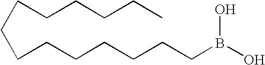 | 89 | 1 |
| 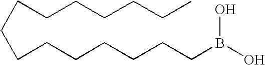 | 90 | |
| 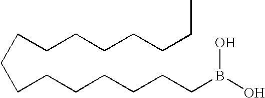 | 62 | |
| 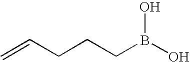 | 72 | 26 |
| 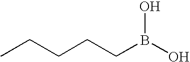 | 42 | |
| 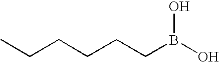 | 53 | |
| 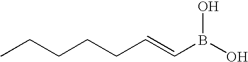 | 63 | |
| 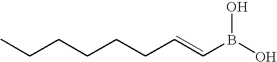 | 77 | |
| 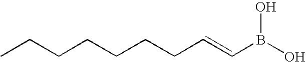 | 82 | |
| 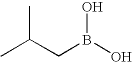 | 9 | |

### V. Lipase Inhibiting Activity of Aromatic Boronic Acids

The endothelial lipase inhibiting activity of various aromatic boronic acids was tested. The results of the assay, which used a 50 micromolar solution of each compound, are shown below. The IC₅₀ of certain compounds for endothelial lipase was also obtained.

| Compound | % Lipase Inhibition | IC₅₀ (micromolar) |
|---|---|---|
| 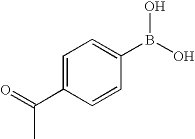 | 80 | 26 |
| 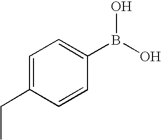 | 83 | 11 |
| 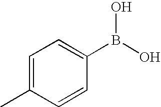 | 88 | 17 |
| 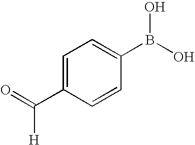 | 60 | |
| 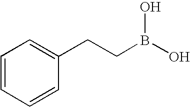 | 25 | |
| 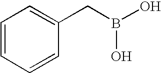 | 10 | |
| 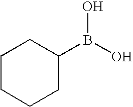 | 1 | |
| 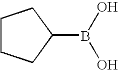 | 7 | |

### Equivalents

Those skilled in the art will recognize, or be able to ascertain using no more than routine experimentation, many equivalents to the specific embodiments of the invention described herein. Such equivalents are intended to be encompassed by the following claims.

All of the above-cited references and publications are hereby incorporated by reference.

1. A compound having the structure of Formula I:
(R¹-L-R²)ₙ(CH₂)ₘ—X (I)
or a pharmaceutically acceptable salt or prodrug thereof, wherein:
R¹ and R² are independently selected from the group consisting of C₁₋₆alkyl, C₁₋₆alkenyl, and C₁₋₆alkynyl;
R is selected from the group consisting of H, C₁₋₆alkyl, and C₁₋₆aralkyl;
L is absent or is selected from the group consisting of O, NR, and S;
X is a functional group that reacts with an active site residue of the targeted lipase to form a covalent adduct;
m is 0 or 1; and
n is an integer from 1-3.

2. The compound of claim 1, wherein X is selected from the group consisting of boronic acid, CN, —SO₂Z¹, P(═O)Z¹, —P(═R³)R⁴R⁵, —C(═NH)NH₂, —CH═NR⁶, and —C(═O)—R;
R³ is O or S;
R⁴ is selected from the group consisting of N₃, SH, NH₂, NO₂, and OYR⁷, and
R⁵ is selected from the group consisting of lower alkyl, amino, OYR⁷, and a pharmaceutically acceptable salt thereof, or
R⁴ and R⁵, together with the phosphorus to which they are attached, form a 5- to 8-membered heterocyclic ring;
R⁶ is selected from the group consisting of H, alkyl, alkenyl, alkynyl, NH₂, —(CH₂)ₚ—R⁷, —(CH₂)_{q}—OH, —(CH₂)_{q}—O-alkyl, —(CH₂)_{q}—O-alkenyl, —(CH₂)_{q}—O-alkynyl, —(CH₂)_{q}—O—(CH₂)ₚ—R⁷, —(CH₂)_{q}—SH, —(CH₂)_{q}—S-alkyl, —(CH₂)_{q}—S-alkenyl, —(CH₂)_{q}—S-alkynyl, —(CH₂)_{q}—S—(CH₂)ₚ—R⁷, —C(O)NH₂, —C(O)OR⁸, and C(Z¹)(Z²)(Z³);
R⁷ is selected from the group consisting of H, alkyl, alkenyl, aryl, heteroaryl, cycloalkyl, cycloalkenyl, and heterocyclyl;
R⁸ is selected from the group consisting of H, alkyl, and alkenyl;
Y is absent or is selected from the group consisting of alkyl, alkenyl, alkynyl, —(CH₂)ᵣ—O—(CH₂)ᵣ—, —(CH₂)ᵣNR₂(CH₂)ᵣ, and —(CH₂)ᵣS(CH₂)ᵣ—;
Z¹ is a halogen;
Z² and Z³ are independently selected from the group consisting of H and halogen;
p is, independently for each occurrence, an integer from 0 to 8;
q is, independently for each occurrence, an integer from 1 to 8; and
r is, independently for each occurrence, and integer from 0-10.

3. The compound of claim 1, wherein X is a group of formula —B(Y¹)(Y²), wherein Y¹ and Y² are independently OH; or —B(Y¹)(Y²) is hydrolysable to a boronic acid.

4. The compound of claim 1, wherein R¹ and R² are independently C₁₋₆alkyl, L is absent, and n is 2.

5. The compound of claim 4, wherein R¹ and R² are independently unsubstituted C₁₋₆ alkyl.

6. The compound of claim 4, wherein m is 0, R¹ is unsubstituted C₃₋₄ alkyl, and R² is unsubstituted C₄ alkyl.

7. The compound of claim 4, wherein m is 1, R¹ is unsubstituted C₃₋₄ alkyl, and R² is unsubstituted C₄ alkyl.

8. A compound having the structure of Formula II: 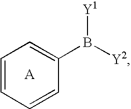
or a pharmaceutically acceptable salt or prodrug thereof, wherein:
Ring A is optionally substituted by one or more functional groups; and
BY¹Y² is B(OH)₂ or a group that is hydrolysable to B(OH)₂, such as a 5- to 8-membered ring that is hydrolysable to a boronic acid.

9. The compound of claim 8, wherein Ring A is substituted by at least one alkyl group.

10. The compound of claim 9, wherein the alkyl group is unsubstituted or substituted by an oxo group.

11. The compound of claim 8, wherein the compound has the structure of Formula III: 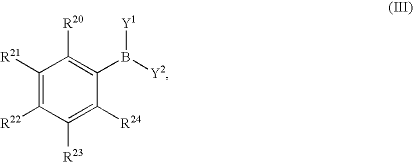
wherein:
R²⁰, R²¹, R²³ and R²⁴ are each independently —H, —COOR′, —CONR′R″, —C(O)R′, —NR′R″, —OH, —SH or a alkyl, alkenyl or alkynyl group optionally substituted by one or more of —COOR′, —CONR′R″, —C(O)R′, —NR′R″, —OH and —SH;
R²² is an unsubstituted C₁₋₁₂ alkyl group or an oxo-substituted C₁₋₁₂ alkyl group;
R′ and R″ are each independently —H or an alkyl, alkenyl, alkynyl, aryl or heteroaryl group; and
BY¹Y² is B(OH)₂ or a group that is hydrolysable to B(OH)₂.

12. The compound of claim 11, wherein three of R²⁰, R²¹, R²³ and R²⁴ are —H and the remaining one of R²⁰, R²¹, R²¹ and R²⁴ is an alkyl, alkenyl or alkynyl group optionally substituted by one or more of —COOR′, —CONR′R″, —C(O)R′, —NR′R″, —OH and —SH.

13. The compound of claim 1, wherein the compound is a lipase inhibitor.

14. The inhibitor of claim 13, wherein the lipase inhibitor inhibits endothelial lipase.

15. A pharmaceutical composition comprising a pharmaceutically acceptable carrier and a compound of claim 1 or 8 or a pharmaceutically acceptable salt or prodrug thereof.

16. A method of inhibiting a lipase in a patient in need thereof, comprising administering to the patient a therapeutically effective amount of a compound of claim 1 or 8.

17. The method of claim 16, wherein the compound increases plasma concentrations of HDL.

18. A method of regulating HDL metabolism in a patient in need thereof, comprising administering to the patient a therapeutically effective amount of a compound of claim 1 or 8.

19. The method of claim 18, wherein the patient is suffering from a vascular disease or condition.

20. The method of claim 19, wherein the vascular disease or condition is selected from the group consisting of angina, atherosclerosis, coronary artery disease, congestive heart failure, hypertension, myocardial infarction, and stroke.

21. The method of claim 18, further comprising administering one or more anti-dyslipidemic agents.

22. The method of claim 21, wherein the one or more anti-dyslipidemic agents are selected from the group consisting of (1) bile acid sequestrants, (2) HMG-CoA reductase inhibitors, (3) HMG-CoA synthase inhibitors, (4) cholesterol absorption inhibitors, (5) acyl coenzyme A-cholesterol acyl transferase (ACAT) inhibitors, (6) cholesteryl ester transfer protein (CETP) inhibitors, (7) squalene synthetase inhibitors, (8) anti-oxidants, (9) PPAR alpha agonists, (10) FXR receptor antagonists, (11) LXR receptor agonists, (12) lipoprotein synthesis inhibitors, (13) renin angiotensin system inhibitors, (14) microsomal triglyceride transport inhibitors, (15) bile acid reabsorption inhibitors, (16) PPAR gamma agonists, (17) triglyceride synthesis inhibitors, (18) transcription modulators, (19) squalene epoxidase inhibitors, (20) low density lipoprotein (LDL) receptor inducers, (21) platelet aggregation inhibitor, (22) 5-LO or FLAP inhibitors, (23) PPAR partial agonists, and (24) niacin or niacin receptor agonists and pharmaceutically acceptable salts and esters thereof.

23. A method of treating metabolic syndrome in a patient in need thereof, comprising administering to the patient a therapeutically effective amount of a compound of claim 1 or 8.

24. The method of claim 23, further comprising administering one or more anti-dyslipidemic agents, anti-diabetic agents, or a combination thereof.

25-28. (canceled)

29. The compound of claim 8, wherein the compound is a lipase inhibitor.

30. The inhibitor of claim 29, wherein the lipase inhibitor inhibits endothelial lipase.
